(19) United States
(12) Patent Application Publication
(10) Pub. No.: US 2021/0205067 A1
(43) Pub. Date: Jul. 8, 2021

(54) PATELLA TENDON REALIGNMENT IMPLANT WITH FIXATION

(71) Applicant: ZKR ORTHOPEDICS, INC., San Francisco, CA (US)

(72) Inventors: Damien SHULOCK, San Francisco, CA (US); Jeffrey HALBRECHT, San Francisco, CA (US); John BARRETT, San Francisco, CA (US); Katherine J. STEPHENSON, Belmont, CA (US)

(21) Appl. No.: 17/127,598

(22) Filed: Dec. 18, 2020

Related U.S. Application Data
(60) Provisional application No. 62/956,555, filed on Jan. 2, 2020.

(51) Int. Cl. A61F 2/08

(52) U.S. Cl. CPC A61F 2/0811; A61F 2002/0858; A61F 2002/0864; A61F 2220/0041; A61F 2002/0829; A61F 2220/0016; A61F 2002/0876

(57) ABSTRACT

An orthopedic implant system including an orthopedic implant with an inferior portion having a surface configured to extend over a top surface of a tibia; a superior portion opposite to the inferior portion and configured to change a position of a patellar tendon by lifting or tilting the patellar tendon when the surface of the inferior portion is engaged with the tibia; a first lateral side disposed between the inferior portion and the superior portion; and a second lateral side disposed between the inferior portion and the superior portion opposite to the first lateral side; and a fixation system to attach the orthopedic implant to the tibia. The invention also includes corresponding methods.

### CROSS-REFERENCE TO RELATED APPLICATIONS

This application claims the benefit of priority of U.S. Provisional Application No. 62/956,555, filed Jan. 2, 2020, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Problems of the patella-femoral joint are a common cause of knee pain. The pain may arise from issues such as poor alignment of the patella or from cartilage breakdown (chondromalacia or arthritis) behind the patella or on the opposing articular surface of the femoral groove (trochlea). Conventional surgical options for treating patella-femoral pain caused by malalignment, chondromalacia or arthritis may include realignment of the patella. For example tracking of the patella may be changed by tilting the patella or by moving the patella to one side or the other. Moving the patella forward (i.e., anteriorly) through a surgical procedure provides another conventional option for treating these conditions. This conventional technique is thought to decrease force across the patella-femoral joint and thus diminish the pain arising from excess force against a worn-out patellar or trochlear cartilage.

Although available, surgical options to realign the patella may be very invasive. For example, surgeries may involve cutting and fixating the bony attachment of the patellar tendon. In particular, conventional techniques may include detaching the patellar tendon from the tibia, then reattaching the patellar tendon at a new location to obtain the desired alignment of the patella. Such invasive surgical techniques may also result in prolonged recovery times. Consequently, an improved mechanism for treating patella-femoral joint problems such as patella-femoral pain, chondromalacia, and/or arthritis is desired.

### SUMMARY OF THE DISCLOSURE

U.S. Pat. No. 9,808,289 discloses a patellar implant configured to be placed between the patellar tendon and the tibia in proximity to the patella to elevate and/or tilt the patellar tendon. While U.S. Pat. No. 9,808,289 discloses some methods and devices for affixing the implant to the tibia, there is a need for other fixation devices and methods that are, e.g., less invasive and possibly less dependent on bone quality. The orthopedic implant systems described herein attach to a patient's tibia and elevate and/or tilt the patellar tendon after implantation.

One aspect of the invention provides an orthopedic implant system with an orthopedic implant with an inferior portion having a surface configured to extend over a tibia, a superior portion opposite to the inferior portion and configured to change a position of a patellar tendon by lifting or tilting the patellar tendon when the curved surface of the first portion is engaged with the tibia, a first lateral side disposed between the inferior portion and the superior portion, and a second lateral side disposed between the inferior portion and the superior portion opposite to the first lateral side; a first clamp engaged with, extending from, the first lateral side, the first clamp comprising a bone engagement edge extending below and beneath the surface of the inferior portion; a second clamp engaged with, and extending from, the second lateral side, the second clamp comprising a bone engagement edge extending below and beneath the surface of the inferior portion; and a tightening element adapted to move the second clamp with respect to the first clamp. In some embodiments, the surface of the inferior portion of the implant is curved. In some or all of the embodiments, the bone engagement edge of the first clamp has teeth and/or the bone engagement edge of the second clamp has teeth.

In some or all of the embodiments according to this aspect of the invention, at least one of the first clamp and the second clamp is removably attachable to the implant. In some such embodiments, the tightening element is adapted to extend from the first clamp to the second clamp, and the tightening element has threads. In some such embodiments, at least one of the first clamp and the second clamp comprises threads corresponding to the tightening element threads. In either or both of these embodiments, the implant may have a passage extending through the implant from the first lateral side to the second lateral side, the tightening element being adapted to be inserted into the passage to extend from the first lateral side to the second lateral side. The passage may be an enclosed passage, or the passage may be a partially enclosed groove.

In embodiments in which at least one of the first clamp and the second clamp is removably attachable to the implant, the tightening element may have first and second screws each adapted to be inserted into a passage through each of the first and second clamps and into the implant. In other embodiments, the tightening element may include a spring extending from the first clamp to the second clamp.

In some embodiments according to this aspect of the invention, the first clamp is irremovably attached to the implant and the second clamp is removably attachable to the implant. In some such embodiments, the tightening element may include a screw adapted to be inserted into a passage through the second clamp and into the implant. In some other such embodiments, the tightening element may include a spring extending from the second clamp to the implant.

In some embodiments according to this aspect of the invention, the tightening element is adapted to extend through a passage in the implant from the first clamp to the second clamp. The passage may be an enclosed passage, or the passage may be a partially enclosed groove. In some or all of these embodiments, the tightening element may have threads. In some or all of these embodiments, the tightening element may have a spring.

Another aspect of the invention provides a method for repositioning a patellar tendon, the method. In some embodiments, the method includes the steps of inserting an orthopedic implant between the patellar tendon and a tibia; engaging an inferior portion of the orthopedic implant with the tibia; changing a position of the patellar tendon with a superior portion of the orthopedic implant system opposite to the inferior portion; and affixing the orthopedic implant to the tibia by engaging a first clamp with a first side of the tibia below the inferior portion and engaging a second clamp of the orthopedic implant system with a second side of the tibia opposite to the first side.

Some embodiments include an additional step of engaging the first clamp with a first lateral side of the orthopedic implant. In either or both of these embodiments, the affixing step may include the step of extending a tightening element through the orthopedic implant from the first clamp to the second clamp. In embodiments in which the tightening element has threads, the affixing step may also include the step of engaging the tightening element threads with threads of the second clamp. In embodiments in which the tightening element includes a spring, the affixing step may include the step of allowing the spring to bias the first clamp toward the second clamp.

In some embodiments according to this aspect of the invention, the affixing step includes the step of inserting a screw through the first clamp into the orthopedic implant. In some such embodiments, the affixing step may also include the step of inserting a screw through the second clamp into the orthopedic implant.

Yet another aspect of the invention provides an orthopedic implant with an inferior portion having a surface configured to extend over a tibia; a superior portion opposite to the inferior portion and configured to change a position of a patellar tendon by lifting or tilting the patellar tendon when the curved surface of the first portion is engaged with the tibia; a first lateral side disposed between the inferior portion and the superior portion; a second lateral side disposed between the inferior portion and the superior portion opposite to the first lateral side; and a fixation blade extending from the surface of the inferior portion, the fixation blade being adapted to penetrate the tibia. In some embodiments, the surface of the inferior portion may be curved. In either or both of these embodiments, the blade may have teeth.

Still another aspect of the invention provides a method for repositioning a patellar tendon. In some embodiments, the method includes the steps of inserting an orthopedic implant between the patellar tendon and a tibia; engaging an inferior portion of the orthopedic implant with the tibia; changing a position of the patellar tendon with a superior portion of the orthopedic implant system opposite to the inferior portion; and affixing the orthopedic implant to the tibia by inserting a blade extending from the inferior portion of the orthopedic implant into the tibia.

Another aspect of the invention provides an orthopedic implant system with an orthopedic implant having an inferior portion having a surface configured to extend over a top surface of a tibia, a superior portion opposite to the inferior portion and configured to change a position of a patellar tendon by lifting or tilting the patellar tendon when the surface of the inferior portion is engaged with the tibia, a first lateral side disposed between the inferior portion and the superior portion, and a second lateral side disposed between the inferior portion and the superior portion opposite to the first lateral side; and a tightening element extending from the first lateral side to the second lateral side, the tightening element adapted to extend around a bottom surface of the tibia opposite to the top surface to engage the inferior portion surface of the orthopedic implant with the tibia with an adjustable engagement force. In some embodiments, the surface of the inferior portion of the implant is curved.

In either or both of these embodiments, the tightening element may include a band. In embodiments in which the band is inelastic, the implant further may have a receptacle adapted to receive a free end of the band. The implant may also have a rotatable screw adapted to draw the free end of the band into the implant to tighten the band. Alternatively, the band may have a plurality of ridges, and the implant may have a ratchet mechanism in the receptacle adapted to end the band to allow insertion of the band into, and prevent withdrawal of the band from, the receptacle.

In some other embodiments, the tightening element is deformable to shorten its effective length.

In embodiments in which the band is elastic, the orthopedic implant may also have a hook or buckle adapted to connect to a free end of the band.

Yet another aspect of the invention provides a method for repositioning a patellar tendon. In some embodiments, the method includes the steps of inserting an orthopedic implant system between the patellar tendon and a tibia; engaging an inferior portion of the orthopedic implant system with the tibia; changing a position of the patellar tendon with a superior portion of the orthopedic implant system opposite to the inferior portion; engaging a tightening element around the tibia from a first lateral side of the implant system to a second lateral side of the implant system; and tightening the tightening element.

In some embodiments, the method also includes the step of inserting a free end of the tightening element into a receptacle in the orthopedic implant. In some such embodiments, the tightening step may include the step of turning a screw in the implant.

In some embodiments, the tightening step may include the step of engaging a free end of the tightening element with a ratchet mechanism in the orthopedic implant. In some embodiments, the tightening step may include the step of deforming the tightening element. In some embodiments, the tightening step may include the step of stretching the tightening element.

Still another aspect of the invention provides an orthopedic implant system with an orthopedic implant having an inferior portion having a surface configured to extend over a tibia, a superior portion opposite to the inferior portion, a first lateral side disposed between the inferior portion and the superior portion; and a second lateral side disposed between the inferior portion and the superior portion opposite to the first lateral side; a frame adapted to extend over the superior portion and the first and second lateral sides; and a fixation element adapted to attach the frame to the tibia, the superior portion and the frame being configured to change a position of a patellar tendon by lifting or tilting the patellar tendon when the surface of the inferior portion is engaged with the tibia.

In some embodiments, the fixation element has a plurality of spikes extending from the frame. In some embodiments, the fixation element has a plurality of screws extending through screw holes in the frame.

Another aspect of the invention provides a method for repositioning a patellar tendon. In some embodiments, the method includes the steps of inserting an orthopedic implant system between the patellar tendon and a tibia; engaging an inferior portion of the orthopedic implant system with the tibia; affixing a frame extending over the superior portion and the first and second lateral sides to the tibia; and changing a position of the patellar tendon with the frame and with a superior portion of the orthopedic implant system opposite to the inferior portion.

In some embodiments, the affixing step includes the step of inserting a plurality of spikes extending from the frame into the tibia. In some embodiments, the affixing step includes the step of inserting a plurality of screws through screw holes of the frame into the tibia.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows an exemplary embodiment of a patellar implant.

FIG. 2 shows another exemplary embodiment of a patellar implant.

FIG. 3 shows another exemplary embodiment of a patellar implant.

FIGS. 4A and 4B show another exemplary embodiment of a patellar implant.

FIGS. 5A and 5B show another exemplary embodiment of a patellar implant.

FIG. 6 shows another exemplary embodiment of a patellar implant.

FIG. 7 shows an exemplary option for placement of an exemplary embodiment of a patellar implant to reposition the patellar tendon and patella.

FIG. 8 shows a patellar implant system with fixation clamps in place on a patient's tibia.

FIG. 9 is a side view of the implant system of FIG. 8.

FIG. 10 is another embodiment of a patellar implant system with fixation clamps.

FIG. 11 is yet another embodiment of a patellar implant system with fixation clamps.

FIG. 12 is still another embodiment of a patellar implant system with fixation clamps.

FIG. 13 is yet another embodiment of a patellar implant system with fixation clamps.

FIG. 14 is another embodiment of a patellar implant system with fixation clamps.

FIG. 15 is an embodiment of a patellar implant system with fixation blades.

FIG. 16 is an embodiment of a patellar implant system with fixation straps.

FIG. 17 is another embodiment of a patellar implant system with fixation straps.

FIG. 18 is an embodiment of a patellar implant system with a fixation frame.

FIG. 19 is another embodiment of a patellar implant system with a fixation frame.

FIG. 20 shows the embodiment of FIG. 19 in place on a patient's tibia.

### DETAILED DESCRIPTION

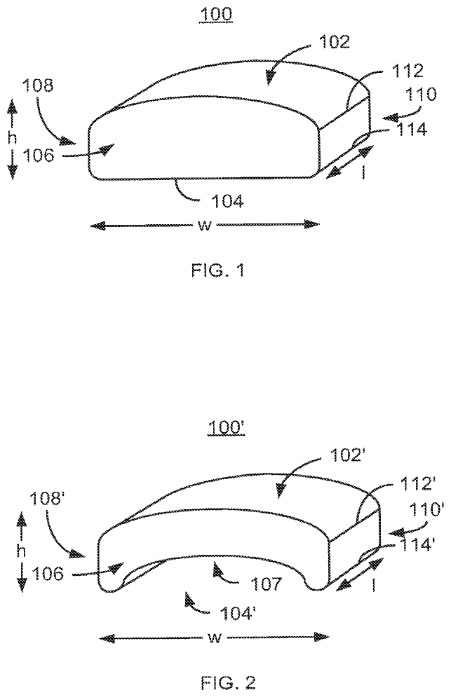

Multiple designs of implants for elevating and/or tilting the patellar tendon are described in U.S. Pat. No. 9,808,289. FIGS. 1-7 show some of the implant designs described in that patent.

Implant 100 in FIG. 1 has a superior portion 102, an inferior portion 104, and an anterior portion 106. The patellar implant 100 includes a posterior portion (not shown) that is opposite to the anterior portion 106. In the embodiment shown, the posterior portion is substantially the same as and parallel to the anterior portion 106. Also shown are lateral sides or surfaces 108 and 110 as well as edges 112 and 114.

The superior portion 102 is shown having a convex upper surface. In addition, the superior portion is symmetric. The superior portion 102 is adapted to reside in proximity to the patellar tendon, while the inferior portion 104 is adapted to reside in proximity to the tibia. The inferior portion 104 of the patellar implant 100 is substantially flat. The superior portion 102 is configured to lift and/or tilt the patellar tendon, while the surface of the inferior portion 104 is configured to fit the tibia. The amount of lift and/or tilt provided may depend upon the shape of the implant 100 as well as the positioning of the implant 100 in the patient. Consequently, the size and/or shape of the implant 100 may vary. For example, in some embodiments, the height, h, of the patellar implant 100 may be between 0.5 and 3 centimeters. In other embodiments, the height may vary between 1 and 2 centimeters. In some embodiments, the width, w, of the patellar implant 100 may be between 1 and 3.5 centimeters. In other embodiments, the width of the patellar implant 100 may vary between 2 and 3 centimeters. Similarly, in some embodiments, the length, I, of the patellar implant may vary between one and 3.5 centimeters. In other embodiments, the length may vary between 1 and three centimeters. In other embodiments, other heights, widths, lengths, and/or angles between components may differ.

In use, the patellar implant 100 may be inserted through an incision in proximity to the patella. The patellar implant 100 is placed below the patellar tendon (not shown in FIG. 1) between the patella (not shown in FIG. 1) and a distal attachment of the patella (not shown in FIG. 1). In other words, the entire patellar implant 100 is placed between the patellar tendon and the tibia and between the patella and a location at which the patellar tendon attaches to the tibia. The location of the patellar implant 100, as well as its particular configuration, is selected to achieve the desired tilt and/or elevation of the patellar tendon and thus the desired repositioning of the patella. In the embodiment shown, the inferior portion 104 is flat. As a result, a slot or flat region may be formed in the tibia at the desired position of the patellar implant 100. The patellar implant 100 is then fitted on the slot such that the inferior portion 104 meets the flat region of the tibia. Once the patellar implant 100 is in the desired position, the patellar implant may be fixed in place, e.g., using one of the fixation mechanisms described below.

FIG. 2 shows another example of a patellar implant 100′. The components of the patellar implant 100′ are analogous to the patellar implant 100 and are, therefore, labeled similarly. Thus, the patellar implant 100′ includes a superior portion 102′, an inferior portion 104′, anterior portion 106′, and posterior portion (not shown in FIG. 2). Also shown are lateral surfaces 108′ and 110′ as well as edges 112′ and 114′.

The superior portion 102′ is shown having a convex upper surface and being symmetric. The superior portion 102′ is still configured to lift and/or tilt the patellar tendon. The superior portion 102′ is still to reside in proximity to the patellar tendon, while the inferior portion 104′ is configured to be placed in proximity to the tibia. The inferior portion 104′ of the patellar implant 100′ has a convex portion 107. In particular, the convex portion 107 is configured to fit the tibia of the patient. As a result, cutting, grinding, or other altering of the tibia in order to fit the implant 100′ may be avoided. Once in place, the patellar implant 100′ may be affixed in place through a variety of mechanisms, described below.

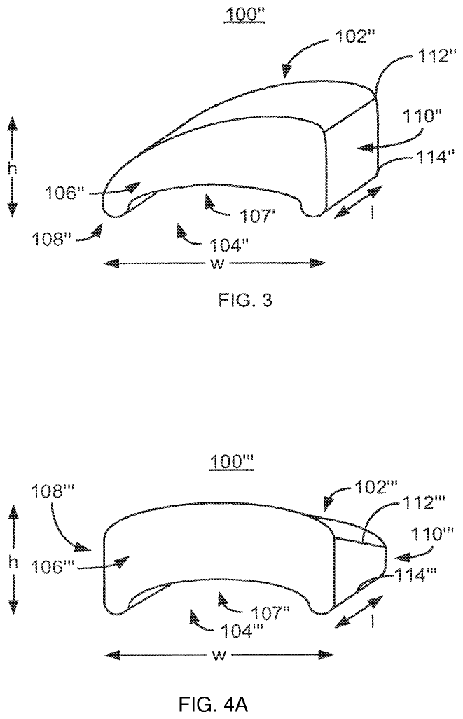

FIG. 3 depicts another exemplary embodiment of a patellar implant 100″. The components of the patellar implant 100″ are analogous to the patellar implants 100 and 100′. Thus, the components of the patellar implant 100″ are labeled similarly. The patellar implant 100″ includes a superior portion 102″, an inferior portion 104″, anterior portion 106″, and posterior portion (not shown in FIG. 3). However, the patellar implant 100″ is wedge shaped. In particular, lateral surface 108/108′ has been replaced with a rounded edge 108″. The lateral surface 110″ is wider than the rounded edge 108″. As a result, the patellar implant 100″ tends to tilt the patellar tendon toward the rounded edge 108″. Consequently, the ability of the patellar implant 100″ to tilt the patellar tendon is improved. A greater tilt of the patellar tendon, as well as the patella, may thus be achieved. Consequently, pain and/or other issues related to patellar-femoral joint problems may be alleviated. Because cutting or other alteration of the tibia may be avoided if the concave portion 107″ is provided, recovery time and ease of use of the patellar implant 100″ may be improved.

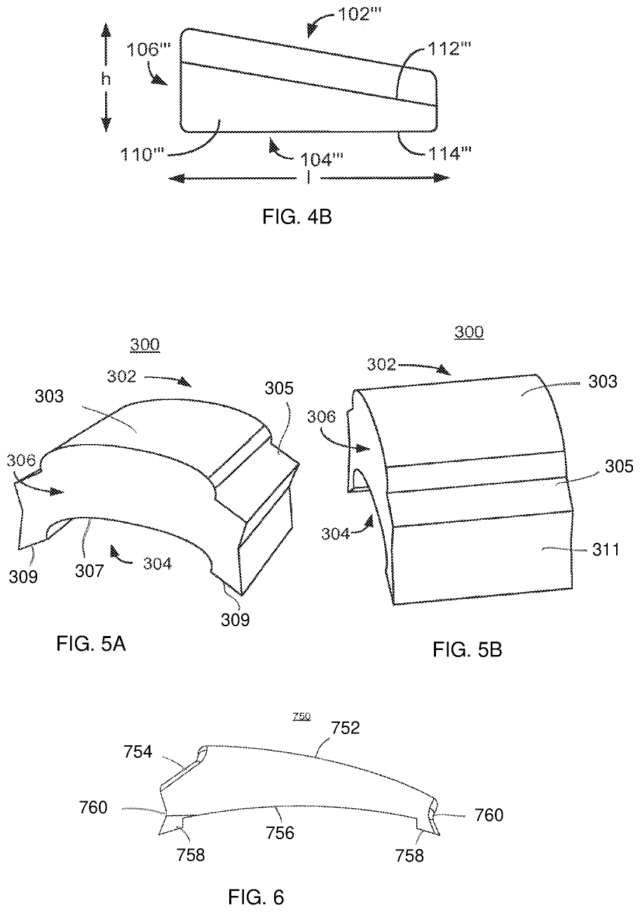

FIGS. 4A and 4B depict perspective and sides views, respectively, of another exemplary embodiment of a patellar implant 100′″. The components of the patellar implant 100′″ are analogous to the patellar implants 100, 100′, and 100″. Thus, the components of the patellar implant 100′″ are labeled similarly. The patellar implant 100′″ includes a superior portion 102′″, an inferior portion 104′″, anterior portion 106′″, and posterior portion (not shown in FIG. 4). However, the patellar implant 100′″ is tapered. In particular, lateral surfaces 108′″ and 110′″ are thicker near the anterior portion 106′″.

The patellar implant 100′″ is used in a similar manner to and made from similar material(s) as the patellar implants 100/100′/100″. The superior portion 102′″ has a convex upper surface and is still configured to lift and/or tilt the patellar tendon. The superior portion 102′″ is still to reside in proximity to the patellar tendon, while the inferior portion 104′″ is configured to be placed in proximity to the tibia. The inferior portion 104′″ has a convex portion 107′″ configured to fit the tibia of the patient. As a result, cutting, grinding, or other altering of the tibia in order to fit the implant 100′″ may be avoided. However, in another embodiment, the concave portion 107′″ may be omitted. Once in place, the patellar implant 100′″ may be affixed in place through a variety of mechanisms, described below.

FIGS. 5A and 5B depict perspective views of another exemplary embodiment of a patellar implant 300. Implant 300 has a superior portion 302 with a curved upper surface 303 configured to be disposed in proximity to the patellar tendon and flanked by cut-outs 305, an inferior portion 304 with a curved surface 307 configured to fit the tibia and flanked by slanted portions 309, an anterior portion 306, and lateral portions 311.

FIG. 6 shows yet another exemplary embodiment of a patellar implant 750. Implant 750 has a superior portion 752 with a curved upper surface configured to be disposed in proximity to the patellar tendon and flanked by a cut-outs 754 on one side, an inferior portion 756 with a curved surface configured to fit the tibia and flanked by slanted portions 758, and lateral portions 760.

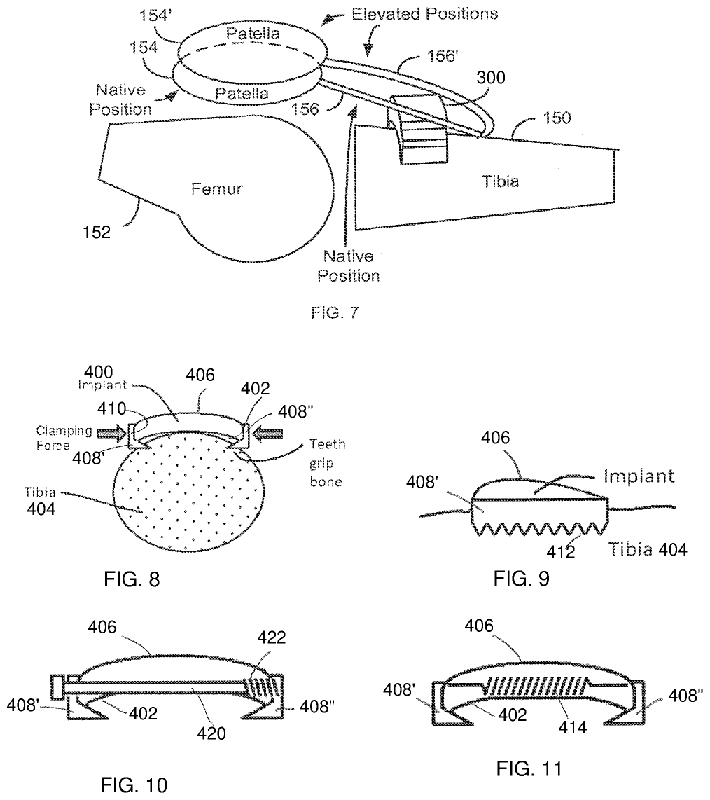

FIG. 7 depicts an exemplary embodiment of one option for placement of an a patellar implant, such as implant 300 from FIGS. 5A-B. For clarity, FIG. 7 is not drawn to scale. For simplicity, only the patellar implant 300 depicted in FIGS. 5A-B is described. However, in other embodiments, other patellar implants described herein may be used. Also shown in FIG. 7 are the tibia 150, femur 152, native-positioned patella 154, elevated-positioned patella 154′, native-positioned patellar tendon 156, and elevated/tilted-positioned patellar tendon 156′. A patient may experience pain and other patellar-femoral joint problems when the patella tendon 156 and patella 154 are in their native positions. The patellar implant 300 is inserted between the patellar tendon 156 and the tibia 150, as well as between the patella 154 and the distal attachment 158 of the patellar tendon 156. The position of the patellar implant 300, as well as the shape and size of the patellar implant 300, may be selected in order to elevate and/or tilt the patellar tendon 156 in the desired manner. For example, for a smaller change in elevation, an implant having a lower height may be selected and/or a patellar implant may be moved closer to the patella 154 and further from the distal attachment 158. In other embodiments, in order to tilt the patellar tendon 156, a wedge shaped patellar implant, such as the implant 100′″ of FIGS. 4A-B. As a result, the patellar tendon 156′ is moved to the elevated/tilted position. The change in position of the patellar tendon 156′ causes a repositioning of the patella 154′ to the elevated/tilted position. Thus, the patella 156′ may be repositioned in order to alleviate pain or other patellar-femoral joint issues.

Any of the implants described above, as well as alternatives to those described above, may be affixed to the tibia. FIGS. 8-14 show embodiments of orthopedic implant systems having a patellar implant and a pair of fixation clamps. In the embodiments of FIGS. 8-14, the orthopedic implant system 400 has an inferior portion 402 configured to engage the tibia 404, a superior portion 406 configured to elevate and/or tilt the patellar tendon (not shown), and a pair of clamps 408′ and 408″ engaged with lateral sides 410 of the implant and with tibia 404. In this embodiment, inferior portion 402 and superior portion 406 each has a curved surface. The clamps 408 extend down from the curved surface of inferior portion 402, and teeth 412 on the downwardly extending portions of clamps 408′ and 408″ grip tibia 404 with a clamping force.

Each of the orthopedic implant system embodiments of FIGS. 8-14 has a mechanism for tightening the clamps so that they grip the tibia. In the embodiment of FIG. 10, a bolt 420 extends through clamp 408′ and through a passage in the implant 400. The passage may be entirely enclosed so that it surrounds the bolt, or it may alternatively be a partially enclosed groove. Threads 422 at the distal end of bolt 420 engage with corresponding threads (not shown) in clamp 408″. Rotation of bolt 420 by the surgeon will move clamps 408′ and 408″ toward each other so that the clamps grip the tibia.

In the embodiment of FIG. 11, clamps 408′ and 408″ are connected by a spring 414 extending through implant 400. Spring 414 biases clamps 408′ and 408″ toward each other to provide a gripping force on the tibia.

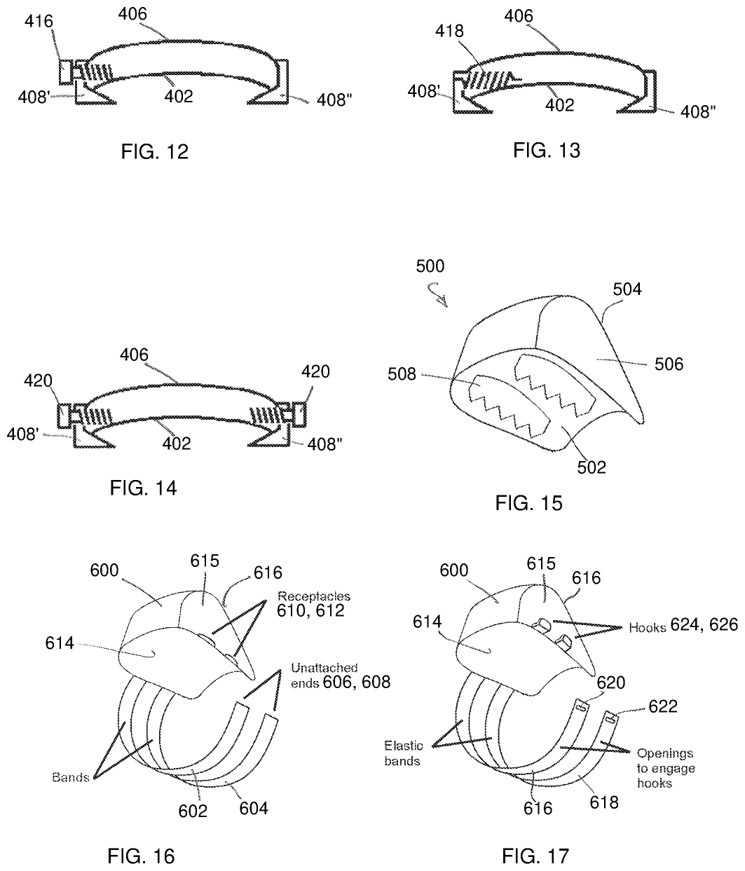

In the embodiment of FIG. 12, clamp 408″ is fixed to the lateral side of the implant 400. After setting clamp 408″ against the surface of the tibia, a screw 416 on the other clamp 408′ may be tightened to cause both clamps to grip the tibia.

In the embodiment of FIG. 13, clamp 408″ is fixed to the lateral side of the implant 400. After setting clamp 408″ against the surface of the tibia, a spring 418 biases clamp 408′ toward the lateral side of the implant, thereby providing a gripping force between the clamps and the tibia.

In the embodiment of FIG. 14, a screw 420 extends through each of clamps 408′ and 408″ into the implant 400 to tighten the clamps, thereby providing a gripping force between the clamps and the tibia.

FIG. 15 shows an orthopedic implant system 500 having an inferior portion 502 with a curved surface configured to engage a tibia, a superior portion 504 configured to elevate and/or tilt a patellar tendon, and lateral side portions 506. In this embodiment, toothed blades 508 extending from inferior portion 502 may be pounded into the bone to affix the implant to the bone.

In some embodiments, such as those shown in FIGS. 16-17, wires, straps, or bands may be wrapped around the tibia and tensioned to the hold the implant in place. The wires, straps, or bands may be made from, e.g., plastic, metal, fabric, webbing, cable, or a combination thereof. The wires, straps, or bands may be used together with other affixation modalities, such as, e.g., the blades of FIG. 15.

FIG. 16 shows an embodiment in which a pair of bands 602 and 604 are attached to, and extend from, one lateral side of implant 600. After placing the curved surface of inferior portion 614 against the top side of the tibia and wrapping around the bands around the tibia, the unattached ends 606 and 608 of the bands are inserted into receptacles 610 and 612, respectively, on the other lateral side 615 of implant 600. The bands may then be tightened using a tightening mechanism. When in place on the tibia, superior portion 616 elevates and/or tilts the patellar tendon.

In one embodiment, the bands are flat and inelastic (e.g., metal), and when inserted into the receptacles, the bands engaged a screw inside the implant. When the screw is turned, the bands are pulled toward the screw, as in a worm drive hose clamp.

In another embodiment in which the bands are flat and inelastic (e.g., metal or plastic), the bands have ridges disposed perpendicular to the bands' long axes. When inserted into the receptacles, the bands' ridges engage a ratchet tooth in or on the implant in the manner of a cable tie to permit only movement of the bands into the receptacles, not movement of the bands out of the receptacles.

In the embodiment of FIG. 17, the bands 616 and 618 are attached to, and extend from, one lateral side of implant 600. The bands are flat and are elastically stretchable along their long axes. After placing the curved surface of inferior portion 614 against the top side of the tibia and wrapping around the bands around the tibia, the bands 616 and 618 are stretched to place the openings 620 and 622 at the free ends of the bands over buckles or hooks 624 and 626 on the other lateral side 615 of implant 600.

In yet another embodiment, the bands may be inelastic but deformable (e.g., a solid metal wire). While under tension, each band can be twisted around itself or around another feature on the implant to tighten the bands, such as with a cerclage wire.

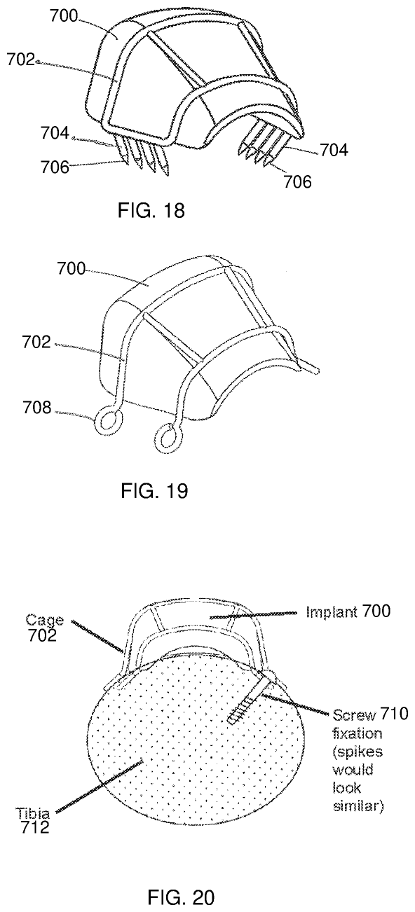

The embodiments of FIGS. 18-20 show yet another approach to affixation of an orthopedic implant to, e.g., a tibia. In FIG. 18, a frame 702 extends over the superior portion and the lateral sides of an implant 700 (such as any of the implants shown in FIGS. 1-6). Frame 702 has fixation elements 704 having sharp tips 706 adapted to penetrate bone. In the embodiment of FIGS. 19-20, frame 702 has rings 708 through each of which a bone screw 710 may be inserted to affix the frame 702 to the patient's tibia 712.

The desired amount of lift and/or tilt of the patellar tendon may vary from patient to patient. Therefore, in any of the embodiments described herein the dimensions of the inferior portion, superior portion and first and second lateral sides may be varied as needed. In addition, in any of these embodiments the lateral sides may be flat or shaped surfaces, or they may be merely the edges formed at the lateral intersections of the inferior and superior portions of the implant.

The patellar implants described herein may include various materials. Particular embodiments may include natural materials, synthetic materials, or a combination thereof. For example, the patellar implant or portions thereof may be constructed using plastics, metals, synthetic bone materials, allografts and/or other materials. Examples of representative materials may include, but are not limited to, stainless steel, titanium, cobalt chrome, polyethylene, polyetheretherketone, and other materials. In some embodiments, the patellar implant 100 may be formed as a composite. For example the superior portion may be made of different materials than the inferior portion. In another embodiment, the superior portion and inferior portion may be made of the same material(s). However, in either embodiment, the superior and inferior portions may be formed as separate pieces, then joined together.

The use of different materials may allow the patellar implant to adapt to the differential forces facing these surfaces. For example, composite patellar implant may include a superior portion fabricated from a polymeric material, with a metal inferior portion. This allows the polymeric material of the superior portion to be in contact with the patellar tendon, while a textured surface on the metal of the inferior portion contacts bone. Other arrangements and combinations might also be used. Coatings, adhesives, surfaces, and/or other features may be used, for example on the surface of the superior portion and/or the surface of the inferior portion. Such treatments may be provided to facilitate local affixation, growth of the tendon and/or bone to the patellar implant, healing, and/or other desired outcomes. As an example, a bone ingrowth surface using metal beads, a bone morphogenic protein, or the like may be provided.

Although depicted with particular shape(s), the patellar implant and/or its components may take any shape that is anatomically acceptable for positioning beneath the patellar tendon and/or patella. For example, edges of the patellar implant may be sharp or rounded. Rounding of edges may reduce stresses placed on the implant during physiological use. Similarly, the surfaces of the implant may be modified to conform to the anatomic position where the implant is to be used. For example, the superior portion may include ridges that may be configured to position or adhere the patellar implant to the portion(s) of the patient's anatomy. In other embodiments, ridges may be omitted.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/− 0.1% of the stated value (or range of values), +/− 1% of the stated value (or range of values), +/− 2% of the stated value (or range of values), +/− 5% of the stated value (or range of values), +/− 10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure.

Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An orthopedic implant system comprising:
an orthopedic implant comprising an inferior portion having a surface configured to extend over a tibia;
a superior portion opposite to the inferior portion and configured to change a position of a patellar tendon by lifting or tilting the patellar tendon when the surface of the first portion is engaged with the tibia;
a first lateral side disposed between the inferior portion and the superior portion; and
a second lateral side disposed between the inferior portion and the superior portion opposite to the first lateral side;
a first clamp engaged with, extending from, the first lateral side, the first clamp comprising a bone engagement edge extending below and beneath the surface of the inferior portion;
a second clamp engaged with, and extending from, the second lateral side, the second clamp comprising a bone engagement edge extending below and beneath the surface of the inferior portion; and
a tightening element adapted to move the second clamp with respect to the first clamp.

2. The system of claim 1 wherein the surface of the inferior portion is curved.

3. The system of claim 1 wherein the bone engagement edge of the first clamp comprises teeth and/or the bone engagement edge of the second clamp comprises teeth.

4. The system of claim 1 wherein at least one of the first clamp and the second clamp is removably attachable to the implant.

5. The system of claim 4 wherein the tightening element is adapted to extend from the first clamp to the second clamp, the tightening element comprising threads.

6. The system of claim 5 wherein at least one of the first clamp and the second clamp comprises threads corresponding to the tightening element threads.

7. The system of claim 5 wherein the implant comprises a passage extending through the implant from the first lateral side to the second lateral side, the tightening element being adapted to be inserted into the passage to extend from the first lateral side to the second lateral side.

8. The system of claim 7 wherein the passage is an enclosed passage.

9. The system of claim 7 wherein the passage is a partially enclosed groove.

10. The system of claim 4 wherein the tightening element comprises first and second screws each adapted to be inserted into a passage through each of the first and second clamps and into the implant.

11. The system of claim 4 wherein the tightening element comprises a spring extending from the first clamp to the second clamp.

12. The system of claim 1 wherein the first clamp is irremovably attached to the implant and the second clamp is removably attachable to the implant.

13. The system of claim 12 wherein the tightening element comprises a screw adapted to be inserted into a passage through the second clamp and into the implant.

14. The system of claim 12 wherein the tightening element comprises a spring extending from the second clamp to the implant.

15. The system of claim 1 wherein the tightening element is adapted to extend through a passage in the implant from the first clamp to the second clamp.

16. The system of claim 15 wherein the passage is an enclosed passage.

17. The system of claim 15 wherein the passage is a partially enclosed groove.

18. The system of claim 15 wherein the tightening element comprises threads.

19. The system of claim 15 wherein the tightening element comprises a spring.

20. A method for repositioning a patellar tendon, the method comprising:
inserting an orthopedic implant between the patellar tendon and a tibia;
engaging an inferior portion of the orthopedic implant with the tibia;
changing a position of the patellar tendon with a superior portion of the orthopedic implant system opposite to the inferior portion; and
affixing the orthopedic implant to the tibia by engaging a first clamp with a first side of the tibia below the inferior portion and engaging a second clamp of the orthopedic implant system with a second side of the tibia opposite to the first side.

21. The method of claim 20 further comprising engaging the first clamp with a first lateral side of the orthopedic implant.

22. The method of claim 20 wherein the affixing step comprises extending a tightening element through the orthopedic implant from the first clamp to the second clamp.

23. The method of claim 22 wherein the tightening element comprises threads, the affixing step further comprising engaging the tightening element threads with threads of the second clamp.

24. The method of claim 22 wherein the tightening element comprises a spring, the affixing step comprising allowing the spring to bias the first clamp toward the second clamp.

25. The method of claim 20 wherein the affixing step comprises inserting a screw through the first clamp into the orthopedic implant.

26. The method of claim 25 wherein the affixing step further comprises inserting a screw through the second clamp into the orthopedic implant.

27. An orthopedic implant comprising:
an inferior portion having a surface configured to extend over a tibia;
a superior portion opposite to the inferior portion and configured to change a position of a patellar tendon by lifting or tilting the patellar tendon when the curved surface of the first portion is engaged with the tibia;
a first lateral side disposed between the inferior portion and the superior portion;
a second lateral side disposed between the inferior portion and the superior portion opposite to the first lateral side; and
a fixation blade extending from the surface of the inferior portion, the fixation blade being adapted to penetrate the tibia.

28. The orthopedic implant of claim 27 wherein the surface of the inferior portion is curved.

29. The orthopedic implant of claim 27 wherein the blade comprises teeth.

30. A method for repositioning a patellar tendon, the method comprising:
inserting an orthopedic implant between the patellar tendon and a tibia;
engaging an inferior portion of the orthopedic implant with the tibia;
changing a position of the patellar tendon with a superior portion of the orthopedic implant system opposite to the inferior portion; and
affixing the orthopedic implant to the tibia by inserting a blade extending from the inferior portion of the orthopedic implant into the tibia.

31. An orthopedic implant system comprising:
an orthopedic implant comprising
an inferior portion having a surface configured to extend over a top surface of a tibia;
a superior portion opposite to the inferior portion and configured to change a position of a patellar tendon by lifting or tilting the patellar tendon when the surface of the inferior portion is engaged with the tibia;
a first lateral side disposed between the inferior portion and the superior portion; and
a second lateral side disposed between the inferior portion and the superior portion opposite to the first lateral side; and
a tightening element extending from the first lateral side to the second lateral side, the tightening element adapted to extend around a bottom surface of the tibia opposite to the top surface to engage the inferior portion surface of the orthopedic implant with the tibia with an adjustable engagement force.

32. The system of claim 31 wherein the surface of the inferior portion is curved.

33. The system of claim 31 wherein the tightening element comprises a band.

34. The system of claim 33 wherein the band is inelastic.

35. The system of claim 34 wherein the implant further comprises a receptacle adapted to receive a free end of the band.

36. The system of claim 35 wherein the implant further comprises a rotatable screw adapted to draw the free end of the band into the implant to tighten the band.

37. The system of claim 35 wherein the band comprises a plurality of ridges, the implant further comprising a ratchet mechanism in the receptacle adapted to end the band to allow insertion of the band into, and prevent withdrawal of the band from, the receptacle.

38. The system of claim 34 wherein the tightening element is deformable to shorten its effective length.

39. The system of claim 33 wherein the band is elastic.

40. The system of claim 39 wherein the orthopedic implant further comprises a hook or buckle adapted to connect to a free end of the band.

41. A method for repositioning a patellar tendon, the method comprising:
inserting an orthopedic implant system between the patellar tendon and a tibia;
engaging an inferior portion of the orthopedic implant system with the tibia;
changing a position of the patellar tendon with a superior portion of the orthopedic implant system opposite to the inferior portion;
engaging a tightening element around the tibia from a first lateral side of the implant system to a second lateral side of the implant system; and
tightening the tightening element.

42. The method of claim 41 further comprising inserting a free end of the tightening element into a receptacle in the orthopedic implant.

43. The method of claim 42 wherein the tightening step comprises turning a screw in the implant.

44. The method of claim 42 wherein the tightening step comprises engaging a free end of the tightening element with a ratchet mechanism in the orthopedic implant.

45. The method of claim 41 wherein the tightening step comprises deforming the tightening element.

46. The method of claim 41 wherein the tightening step comprises stretching the tightening element.

47. An orthopedic implant system comprising:
an orthopedic implant comprising an inferior portion having a surface configured to extend over a tibia;
a superior portion opposite to the inferior portion;
a first lateral side disposed between the inferior portion and the superior portion; and
a second lateral side disposed between the inferior portion and the superior portion opposite to the first lateral side;
a frame adapted to extend over the superior portion and the first and second lateral sides; and
a fixation element adapted to attach the frame to the tibia, the superior portion and the frame being configured to change a position of a patellar tendon by lifting or tilting the patellar tendon when the surface of the inferior portion is engaged with the tibia.

48. The system of claim 47 wherein the fixation element comprises a plurality of spikes extending from the frame.

49. The system of claim 47 wherein the fixation element comprises a plurality of screws extending through screw holes in the frame.

50. A method for repositioning a patellar tendon, the method comprising:
inserting an orthopedic implant system between the patellar tendon and a tibia;
engaging an inferior portion of the orthopedic implant system with the tibia;
affixing a frame extending over the superior portion and the first and second lateral sides to the tibia; and
changing a position of the patellar tendon with the frame and with a superior portion of the orthopedic implant system opposite to the inferior portion.

51. The method of claim 50 wherein the affixing step comprises inserting a plurality of spikes extending from the frame into the tibia.

52. The method of claim 50 wherein the affixing step comprises inserting a plurality of screws through screw holes of the frame into the tibia.
